(19) United States
(12) Patent Application Publication
(10) Pub. No.: US 2014/0228607 A1
(43) Pub. Date: Aug. 14, 2014

(54) FURNACE SYSTEM AND OPERATION INCLUDING FUEL GAS SUPPLY TO FURNACE BURNERS

(71) Applicant: Equistar Chemicals, LP, Houston, TX (US)

(72) Inventor: Richard B. Halsey, Houston, TX (US)

(73) Assignee: Equistar Chemicals, LP, Houston, TX (US)

(21) Appl. No.: 14/176,642

(22) Filed: Feb. 10, 2014

Related U.S. Application Data
(60) Provisional application No. 61/764,799, filed on Feb. 14, 2013.

(51) Int. Cl. C07C 5/03; F23D 99/00

(52) U.S. Cl. CPC C07C 5/03; F23D 21/005
USPC 585/256; 422/129; 431/159

(57) ABSTRACT

An olefin production system having a pyrolysis furnace that is configured to thermally crack hydrocarbon feedstock into olefins and a fuel gas system having a hydrotreater system that is configured to hydrogenate process gas. The pyrolysis furnace has a burner and the fuel gas system is configured to supply the hydrogenated process gas as fuel gas to the burner.

This application claims priority to U.S. Provisional Application No. 61/764,799, filed on Feb. 14, 2013, the contents of which are incorporated herein by reference in its entirety.

### BACKGROUND

1. Field

The presently disclosed technique relates generally to a furnace system and operation including a system and method to process furnace fuel gas and, more specifically, to hydrotreat fuel gas supply to burners of the furnace.

2. Description of Related Art

This section introduces information that may be related to aspects of the presently disclosed technique that is described and/or claimed below. This discussion is believed to be helpful in providing the reader with background information to facilitate a better understanding of the various aspects of the presently disclosed technique. Accordingly, it should be understood that these statements are to be read in this light, and not as admissions of prior art.

Process gas, such as certain process vent or waste streams from unit operations in chemical plants and refineries, may find other uses in the process. For example, it may be used as fuel gas to recover the heating value of the process gas, to reduce waste, to avoid emission of those streams to the environment, and so forth. A fuel gas system may receive process gas streams and provide the process gas as fuel gas to burners of a furnace. A furnace may provide heat to an incoming primary feed via one or more burner flames. The burners combust a fuel (e.g., fuel gas) in the presence of air or oxygen to generate the burner flames. Reaction products of the combustion in the burner may flow through the furnace and discharge from the furnace as an exhaust gas or flue gas.

Unfortunately, combustion of fuel at the burners may foul the burner over time. Fouling of the furnace burners (and burner tips) generally reduces the effectiveness of the furnace. In operation, fouled burners may cause inefficient and incomplete combustion of fuel and, therefore, waste fuel, reduced heat generation (and thus reduce heat transfer to the furnace primary feed), increase undesirable components in the flue gas, and so forth. In all, operation of fouled furnace burners can negatively impact furnace economics, furnace product quality, flue gas composition, and so on.

Certain burner designs may be more susceptible to fouling and/or to problematic operation upon fouling. For, example, burners and their tips designed to reduce nitrogen oxide (NOx) emissions in the furnace flue gas discharged to the atmosphere, may require that the burners and burner tips remain generally clean and not fouled for effective operation. These low NOx burners may be important to satisfy stringent federal, state, and municipal regulations that impose maximum total NOx concentration limits on streams discharged to the environment. Fouled low NOx burners may result undesirably in increased NOx emissions in the furnace flue (exhaust) gas.

The presently disclosed technique is directed to resolving, or at least reducing, one or more of the problems mentioned above. Even if solutions are available to the art to address these issues, the art is always receptive to improvements or alternative means, methods and configurations. Thus, there exists and need for technique such as that disclosed herein.

### SUMMARY

One aspect of the invention relates to an olefin production system including: a pyrolysis furnace to thermally crack hydrocarbon feedstock into olefins; and a fuel gas system comprising a hydrotreater to hydrogenate process gas for fuel gas supply to burners of the pyrolysis furnace.

Another aspect of the invention relates to a furnace system including a furnace, and a hydrotreater to hydrogenate unsaturated compounds in fuel gas supplied to a burner of the furnace.

Yet another aspect of the invention relates to a method of producing olefins, the method including: receiving feedstock and thermally cracking the feedstock in a pyrolysis furnace to give olefins; and receiving process gas and hydrotreating the process gas for supply as fuel gas to a burner of the pyrolysis furnace.

Yet another aspect of the invention relates to a fuel system for one or more furnaces, including: a process fluid header configured to receive process fluid; and a hydrotreater reactor configured to hydrogenate unsaturated hydrocarbons in the process fluid to give a treated process fluid, wherein the fuel system is configured to supply the treated process fluid as fuel to a burner of a furnace.

The above presents a simplified summary of the presently disclosed subject matter in order to provide a basic understanding of some aspects thereof. The summary is not an exhaustive overview, nor is it intended to identify key or critical elements to delineate the scope of the subject matter claimed below. Its sole purpose is to present some concepts in a simplified form as a prelude to the more detailed description set forth below.

### BRIEF DESCRIPTION OF THE DRAWINGS

The invention may be understood by reference to the following description taken in conjunction with the accompanying drawings, in which like reference numerals identify like elements, and in which:

FIG. 1 is an exemplary furnace system in accordance with embodiments of the present techniques.

FIG. 2 is an example of the fuel gas system in the furnace system of FIG. 1 in accordance with embodiments of the present techniques.

FIG. 3 is an example of the hydrotreater system of the fuel gas system FIG. 1 and FIG. 2 in accordance with embodiments of the present techniques.

FIG. 4 is an exemplary method of operating a furnace system in accordance with embodiments of the present techniques.

While the invention is susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION

One or more specific embodiments of the presently disclosed technique will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill in the art and having the benefit of this disclosure.

The presently disclosed techniques recognize that the burner fouling may result because the burner tips are exposed to the high temperatures in the furnace. In particular, this high heat may cause the unsaturated components, such as olefins (and diolefins) which have a reactive double bond, to polymerize before the unsaturated components can combust or burn. Various embodiments hydrogenate unsaturated compounds in the process gas received as fuel gas and sent to burner(s) of an industrial furnace. An example of the industrial furnace is a pyrolysis furnace that produces olefins. Again, the process gas (e.g., process vent gas, process waste gas, etc.) to be used as fuel gas may be received from a chemical plant or refinery, for example. The techniques employ a hydrotreater to perform the hydrogenation of the process gas.

In accordance with the presently disclosed techniques, these unsaturated compounds such as olefins, diolefins, and acetylenes may be converted into a non-polymerizing fuel by saturating the olefin and alkyne bonds to single bonds by reacting with hydrogen. This reaction can be carried out at moderate temperatures and hydrogen partial pressures using a hydrotreater reactor with hydrogenation catalyst. This hydrogenation of the process gas may reduce fouling in the furnace burners and be especially beneficial with low NOx burners, for instance, which may require clean and non-fouled burner tips to function effectively or properly.

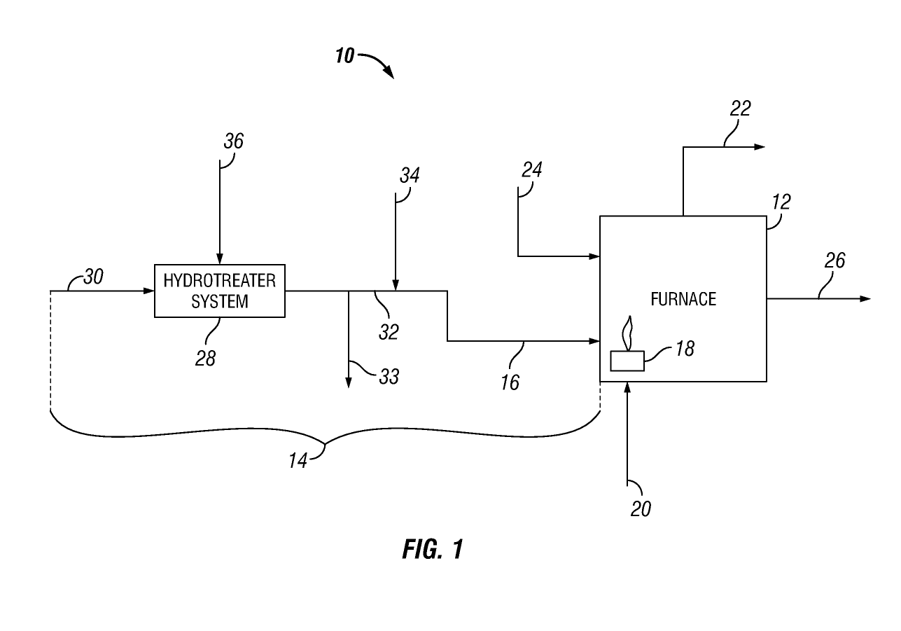

FIG. 1 is a furnace system 10 having a furnace 12 and a fuel gas system 14. The furnace 12 may be a steam boiler or electricity generator, or a furnace in olefin production or other chemical plant applications, refinery applications, and so on. The fuel gas system 14 supplies fuel gas 16 to one or more burners 18 of the furnace 12. The burners 18 may be disposed on the floor or walls of the furnace 12, for example. Air 20 or oxygen may be fed to the burners 18 to facilitate combustion of the fuel gas 16 through the burners 18. The products of the combustion of the fuel gas 16 may discharge (e.g., overhead) from the furnace 12 as vent gas or flue gas 22. The flue gas 22 may be treated (not shown) after discharge from the furnace 12 and prior to being discharged to the atmosphere, for example.

The flame emitting from the burners 18 may heat the primary feed 24 routed (e.g., in conduits or tubes, fire or radiant boxes, etc.) through the furnace 12. This primary feed 24 heated in the furnace 12 may exit the furnace as product 26 of the furnace 12. The product 26 may be further processed downstream of the furnace 12. Moreover, the primary feed 24 entering the furnace 12 may be preheated (not shown) such as in a steam heat exchanger, and/or additionally processed such as subjected to vaporization, upstream of the furnace 12 or in a forward portion of the furnace 12. In certain embodiments, the nature and composition of the primary feed 24 is not material to the practice of the technique and will be implementation-specific.

In the case of olefin production, the primary feed 24 may be a hydrocarbon feedstock, for instance. Heat from the flames of the burners 18 facilitates thermal cracking of the hydrocarbon feedstock flowing through tube banks in the furnace 12, for example. The thermally-cracked hydrocarbon feedstock has olefins and discharges as product 26 of the furnace 12. Of course, again, the furnace 12 may instead be an application in refinery treatments, steam generation, electricity generation, and so on.

The fuel gas system 14 includes a hydrotreater system 28 that treats process gas 30 (e.g., waste gas, vent gas, etc.) to give treated process gas 32 to be supplied as fuel gas 16 to the furnace 12. The fuel gas system 14 may supplement the treated process gas 32 with natural gas 34, methane, or other fuel, to give the fuel gas 16 supply to the burners 18. The natural gas 34 or other fuel may be added at other locations in lieu of or in addition to the location depicted in FIG. 1. For example, the natural gas or other fuel may be added upstream of the hydrotreater system 28 or in the hydrotreater system 28.

Moreover, in certain embodiments, the treated process gas 32 may be supplied to burners in additional furnaces (not shown), as indicated by reference numeral 33. Indeed, in embodiments, the fuel gas system 14 is a general fuel gas system at a chemical plant or refinery, for example. Such a fuel gas system 14 may collect process gas 30 (e.g., process waste gas or vent gas, etc.) from various sources (unit operations, storage areas, etc.), hydrotreat the process gas 30 via hydrotreater system 28, and distribute the treated process gas 32 as fuel gas to a plurality of furnaces at the facility, including to the depicted furnace 12 and other furnaces (not shown). Moreover, low NOx burners have become more common in various furnaces at a facility. The present techniques may facilitate adequate operation of low NOx burners and thus help to reduce NOx emissions.

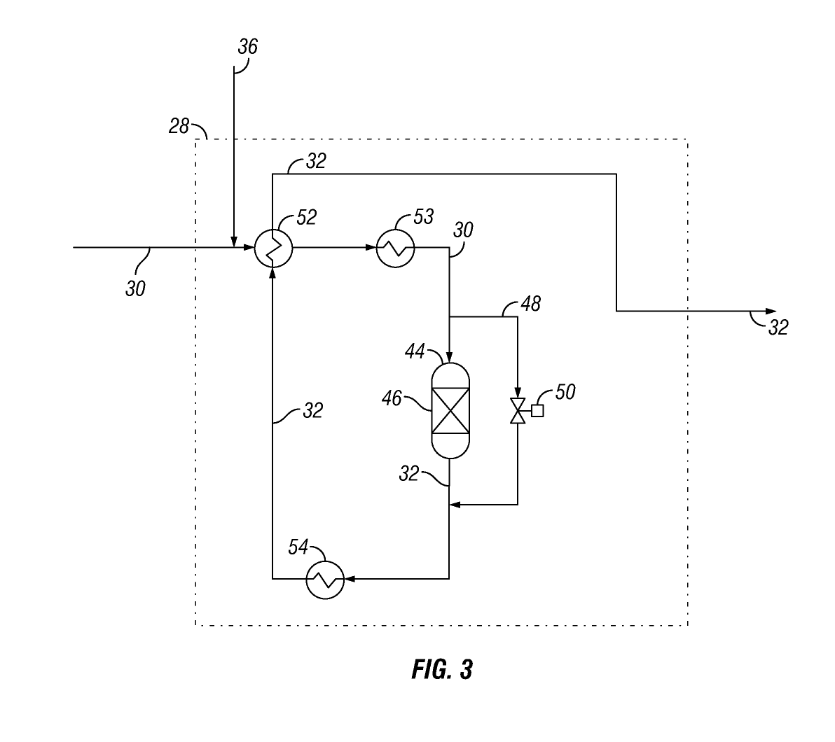

The hydrotreater system 28, or the hydrotreater reactor (see reactor 44 of FIG. 3) in the hydrotreater system 28, may be generally referred to as a hydrotreater. The hydrotreater system 28 hydrogenates unsaturated compounds in the process gas 22. In certain examples, an adequate amount of hydrogen exists in the incoming process gas 30 for the hydrogenation. In other examples, hydrogen 36 may be added for the hydrogenation, such as if the incoming process gas 30 has no hydrogen or an inadequate amount of hydrogen.

As indicated, the upstream process gas 30 fed to the hydrotreater system 28 may be collected from one or more sources. Moreover, the fuel gas system 14 may generally include a process gas collection system disposed upstream of the hydrotreater system 28. The sources of the process gas 30 (e.g., process waste gas, vent gas, etc.) may exist at the same facility as the furnace system 10, or at a facility remote to the furnace system 10. The sources of process gas 30 may include processes, systems, or unit operations in a chemical plant, refinery, storage facility, and so forth.

In certain embodiments, the furnace system 10 may be further integrated with the facilities (chemical plant, refinery, or storage area, etc.) providing process gas. In other words, the plant or storage area providing process gas 30 may also provide the primary feed 24 to the furnace 12 and/or use the furnace 12 product 26, and the like.

For example, if the furnace 12 is a pyrolysis furnace in olefin production, an upstream refinery may process gas for fuel gas, and also provide hydrocarbon feedstock as primary feed 24 to the furnace 12. In another example, if the furnace 12 is a steam boiler, for instance, a refinery or chemical plant may provide process gas for fuel gas, use product 26 steam generated by the steam boiler, and return steam condensate for boiler feedwater as primary feed 26. However, in other examples, the facilities as sources of the process gas 30 are not further integrated with the furnace system 10.

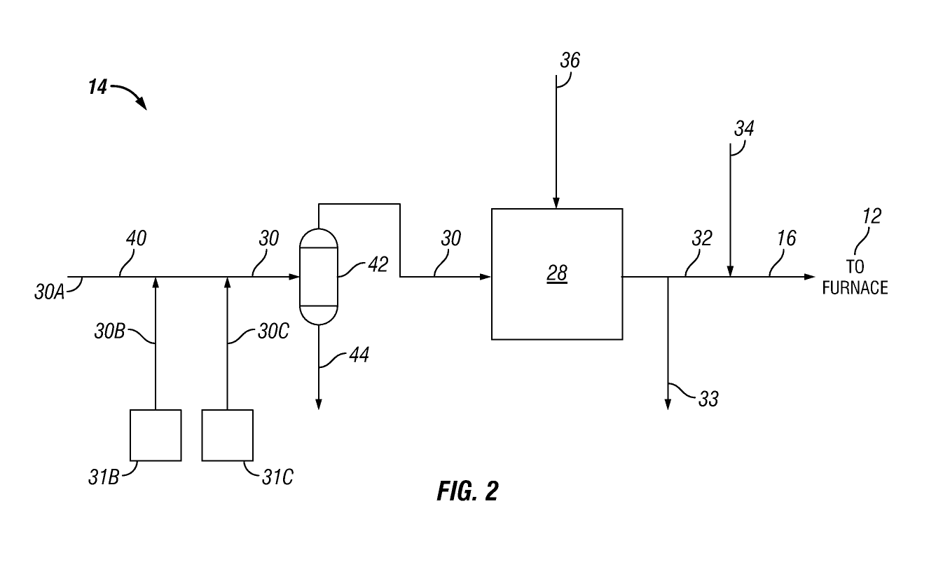

FIG. 2 is an example of one particular implementation of the fuel gas system 14 of FIG. 1. The fuel gas system 14 may be a dedicated system to the furnace 12 of FIG. 1, or may be a general system supplying fuel gas to multiple consumers of fuel gas at a production facility.

In some embodiments, process gas 30A, 30B, 30C may be collected from various sources via individual lines culminating in a process gas header 40, for example, to give the total process gas 30 sent to the hydrotreater system 28. The depicted process gas 30A may be from a single source or may by process gas combined from more than one source. Sources 31B and 31C (e.g., a refinery, a chemical plant, a storage system, process equipment downstream of the furnace, etc.) provide the process gas 30B and 30C, respectively. In the illustrated embodiment, the process gas 30 is routed through a knock-out pot 42 to remove small amounts of entrained liquid 44 that might exist in the process gas 30.

Any entrained liquid 44 removed (e.g., via a simple physical separation in the knock-out pot 42) discharges from a bottom portion of the knock-out pot 42, for example. The process gas 30 discharges overhead from the knock-out pot 42 to the hydrotreater system 28. In certain embodiments, a process gas collection system may include the process gas header 40, the individual lines or subheaders feeding the process gas header 40, the knockout pot 42, the feeding of process gas to the hydrotreater system 28 from the overhead of the knock out pot 42, and so on. It should be noted that other embodiments of the fuel gas system 14 do not include a knock-out pot 42 for the process gas 30. Moreover, the exemplary configuration generally given in FIG. 2 is not meant to limit the all the claims presented below. Indeed, a variety of configurations for collecting, receiving, treating, and managing process gas are contemplated.

The hydrotreater system 28 hydrogenates unsaturated components in the process gas 30. Thus, the hydrotreater system 28 converts unsaturated hydrocarbons (e.g., olefins, acetylenes) in the process gas 30 into saturated hydrocarbons (e.g., alkanes) in the treated process gas 32. As mentioned, hydrogen 36 may be added to the hydrotreater system 28 if needed for the hydrogenation. Moreover, as also mentioned, natural gas 34 or other fuel may be added to supplement the treated process gas 32 to give the fuel gas 16 sent to the furnace 12 burners 18. Lastly, in some examples, the treated process gas 32 may be supplied to burners in additional furnaces, as indicated by reference numeral 33.

Many process gas 30A, 30B, 30C streams discharging from refineries and chemical plants to a fuel gas system 14 may contain substantial amounts of hydrogen. In certain examples, the hydrogen content of the process gas 30 is greater than the amount of the combined unsaturated components (e.g., combined olefins, diolefins, acetylenes) on a molar unsaturated (olefin, alkyne) bond basis. Therefore, as previously mentioned, hydrogen may not need to be added to the existing process gas 30 for the hydrogenation reaction in the hydrotreater system 28.

Beneficially, the lack of a need for hydrogen addition may reduce capital and operating costs of the presently disclosed techniques in some embodiments. An additional benefit in some embodiments is that reacting the contained hydrogen in the process gas 30 with the existing olefins in the process gas 30 beneficially reduces the amount of free hydrogen content of the fuel gas 16 stream. Free hydrogen can undesirably increase localized temperatures in the downstream furnace 12 that may increase NOx production, for instance.

If adequate hydrogen is typically not present in the process gas 30, hydrogen may be readily available at many facilities for addition. Whether hydrogen is available onsite or brought onsite, hydrogen 36 can be added to the hydrotreater system 28. Such hydrogen addition to the hydrotreater system 28 may be implemented with the appropriate flow control equipment to meet the hydrotreating and furnace process requirements.

The amount of olefins, diolefins, and acetylenes in the process gas 30 can vary widely depending upon the particular source of the fuel gas. Yet, generally, the total concentration of combined olefins, diolefins, and acetylenes may be present at reasonably low but undesirable concentration levels that can range upwardly to about 5 vol %. Typically, the olefins, diolefins, and acetylenes concentration of the fuel gas stream in embodiments can be in the range of from 1 part per million by volume (ppmv) to 3 vol %, and, more typically, it is in the range of from 2 ppmv to 1 vol %. Of course, the present techniques may accommodate combined olefins, diolefins, and acetylenes concentrations outside of these numerical ranges.

The presently disclosed techniques may be particularly useful in the processing of refinery process gas 30A, 30B, 30C streams that are from any one or more of the numerous process units of a crude oil refinery. These refinery process gas streams may separately be introduced into the hydrotreater reactor (not shown) of the hydrotreater system 28, or combined and introduced as one or more combined feeds into the hydrotreater reactor of the hydrotreater system 28. The illustrated embodiments of FIG. 1 and FIG. 2 depict the process gas 30 fed to the hydrotreater system as a combined stream.

FIG. 3 is an example of the hydrotreater system 28 of FIG. 1 and FIG. 2. The process gas 30 is routed through a hydrotreater reactor 44 having one or more beds of hydrogenation catalyst 46. A bypass 48 may divert the process gas 30 around the hydrotreater reactor 44 in response to excessive temperature in the hydrogenation reactor 44, for example. In certain embodiments, the bypass valve 50 operates normally closed, and opens automatically in response to excessive temperature sensed in the hydrogenation reactor 44.

The unsaturated hydrocarbons (e.g., olefins, diolefins, acetylenes) in the process gas 30 are hydrogenated as the process gas 30 flows across the hydrogenation catalyst 46 in the hydrogenation reactor 44. The unsaturated hydrocarbons may become fully saturated depending on the conditions of the hydrogenation. Of course, some amount of unsaturated hydrocarbons may remain in the treated process gas 32.

The treated process gas 32 discharges from the hydrotreater reactor 46 and is routed through a heat recovery exchanger 52 in this embodiment. The treated process gas 32 will typically be greater in temperature than the incoming process gas 30 because the hydrogenation reaction in the hydrotreater 44 is generally exothermic. Therefore, the warmer treated process gas 32 may be cross-exchanged with the incoming cooler process gas 30 to heat the incoming process gas 30. Advantageously, such heating may bring the temperature of the incoming process gas 30 closer to the hydrogenation reaction temperature in the hydrotreater reactor 44. Moreover, during startup, a heat exchanger 53 (e.g., shell and tube heat exchanger) may have steam or steam condensate on the utility side and be temporarily operated to heat the incoming process gas 30 until the hydrogenation reaction in the hydrotreater 44 is initiated and maintained.

Further, prior to the thermal cross-exchange between the incoming process gas 30 and the treated process gas 32, the treated process gas 32 may first flow through another heat exchanger 54, as depicted in the illustrated embodiment. Similar to startup heater 53, this heat exchanger 54 may also function as a startup heater, for example, using steam, steam condensate, or other heating medium to heat the treated process gas 32. As indicated, the process gas 30 may need to be heated until the hydrogenation reaction in the hydrotreater reactor 44 is initiated. In other words, during startup of the hydrotreater reactor 44 and prior to initiation of the hydrotreating reaction, the process gas 30 flowing through hydrotreater reactor 44 is heated by the heat exchanger 54. The heated process gas is then cross-exchanged with the incoming process gas 30 via the heat recovery exchanger 52 to heat the incoming process gas 30. Yet, again, the heat exchanger 53 may be used to directly heat the process gas 30.

The heat exchanger 54 on the discharge of the hydrotreater 44 may additionally function as a trim cooler, for example, and which employs a cooling medium (e.g., cooling tower water) on the utility side of the heat exchanger 54. Therefore, after the hydrogenation reaction is initiated and the hydrogenation reaction continuing in the hydrotreater reactor 44, the heat exchanger 54 may switch in operation from a startup heater to a trim cooler to remove some of the exothermic heat of reaction absorbed by the treated process gas 32. Thus, in normal operation and not in startup, the heat exchanger 54 may operate as a cooler or trim cooler. Such trim cooling of the treated process gas 32 may beneficially prevent too much heat from being added to the incoming process gas 30 at the heat recovery exchanger 52 (cross-exchanger) and thus reduce the likelihood of excessive temperature in the hydrotreater reactor 44.

As indicated, the hydrotreating step may be performed with a hydrotreater reactor 44, which may include a reactor vessel that defines a volume and which contains one or more beds of hydrotreating (hydrogenation) catalyst. The process gas 30 or fuel gas stream is introduced into the hydrotreater reactor 44 wherein it is contacted with the hydrotreating catalyst. In certain examples, the reaction conditions within the reactor vessel are maintained so to saturate olefins, diolefins, and acetylenes.

The hydrotreating (hydrogenation) catalyst 46 may form one or more beds of catalyst in the hydrotreater reactor 44. The hydrotreating catalyst 46 can be a variety of hydrogenation catalysts including conventional hydrotreating catalysts that have a metal component on a support material. A typical supported metal is palladium or nickel. The metal component can include a Group VIB metal component or a Group VIIIB metal component, or both a Group VIB metal component and a Group VIIIB metal component. The hydrotreating catalyst can also include a promoter such as a phosphorous, bismuth, silver or gold component, and so forth.

The amount of Group VIIIB metal in the hydrotreating catalyst 46 composition can be in the range of from about 0.05 to about 10 weight percent elemental metal based on the total weight of the hydrotreating (hydrogenation) catalyst composition. More narrow ranges of the concentration of Group VIII metal in the hydrotreating catalyst composition may include the range of from 0.1 weight % to 8 weight %, and in the range of from 0.2 weight % to 5 weight %. The metals can be in a reduced, oxide, or sulfided state, and so forth

The support material of the hydrotreating (hydrogenation) catalyst 46 may provide a support for the metal hydrogenation components of the hydrotreating catalyst 46. The support may be porous refractory oxides and other materials. Examples of porous refractory oxides include silica, magnesia, silica-titania, zirconia, silica-zirconia, titania, titania-alumina, zirconia-alumina, silica-titania, alumina, silica-alumina, and alumino-silicate. The alumina can be of various forms, such as, alpha alumina, beta alumina, gamma alumina, delta alumina, eta alumina, theta alumina, boehmite, or mixtures thereof. In certain examples, the porous refractory oxide used as a support is gamma alumina.

The porous refractory oxide may have an average pore diameter in the range of from about 30 Angstroms to about 500 Angstroms, from 50 Angstroms to 400 Angstroms, or from 60 Angstroms to 300 Angstroms. The total pore volume of the porous refractory oxide, as measured by standard mercury porosimetry methods, may be in the range of from about 0.2 cubic centimeter (cc)/gram to about 2 cc/gram, from 0.3 cc/gram to 1.5 cc/gram, or from 0.4 cc/gram to 1 cc/gram. The surface area of the porous refractory oxide, as measured by the Brunauer-Emmett-Teller Method (B.E.T.) method, generally exceeds about 50 square meter (m2)/gram, and it is typically in the range of from about 100 m2/gram to about 500 m2/gram.

The temperature and pressure conditions within the hydrotreater reactor 44 vessel may be controlled to provide desired reaction conditions for the hydrogenation of olefin and alkyne saturation to saturated hydrocarbon. The contacting temperature may generally be in the range of from 85° F. to 896° F. More narrow ranges include from 95° F. to 842° F., and from 100° F. to 650° F. As for the contacting pressure, it may generally be in the range of from 20 pounds per square inch (psig) to 600 psig. More narrow ranges include from 30 psig to 500 psig, and from 70 psig to 400 psig.

The flow rates at which the process gas 30 or fuel gas stream is charged to the hydrotreater reactor 44 vessel may provide a gaseous hourly space velocity (GHSV) in the range of from 0.01 hr−1 to 6000 hr−1, for example The term “gaseous hourly space velocity,” as used herein, means the numerical ratio of the rate at which the process gas 30 stream, including added hydrogen, if any, that is charged to the hydrotreater reactor 44 vessel in volume (at standard temperature and pressure conditions) per hour divided by the volume of hydrotreating catalyst 46 in the reactor vessel to which the process gas or fuel gas stream is charged. Examples of GHSV are in the range of from 0.05 hr−1 to 4000 hr−1, from 0.1 hr−1 to 3500 hr−1, and from 0.2 hr−1 to 3200 hr−1.

In certain examples, typical refinery gas streams that are to be process gas 30 feed streams to the hydrotreater reactor of the present techniques may be those generated by a delayed coker unit such as the coker dry gas and coker propylene vapor, a fluid catalytic cracking (FCC) unit such as the FCC dry gas, a flare gas recovery system, tank vents, vapor overheads from crude unit atmospheric and vacuum towers, and the like. The process gas 30 streams yielded from these process units can have significant concentrations of olefinic compounds. The refinery process gas 30 streams (to be used as fuel gas 16) can include significant concentration levels of light or lower olefin compounds such as ethylene, propylene, butenes, and pentenes, and also smaller amounts of acetylenes. Typical concentration ranges for these light olefins and acetylenes in the refinery gas streams may fall in the concentration ranges listed above.

As indicated, embodiments of the present techniques are applicable in olefin production in which the furnace 12 is a steam cracking unit that receives primary feed 24 of ethane through gas oils to produce ethylene and propylene and recovery of heavy byproducts. Moreover, process gas streams discharging from downstream processing of a steam cracking furnace 12 product 26 may be used as fuel gas, and may be similar to those process gas streams discharging from a refinery and also contain light olefins byproducts that can foul furnace 12 burners 18.

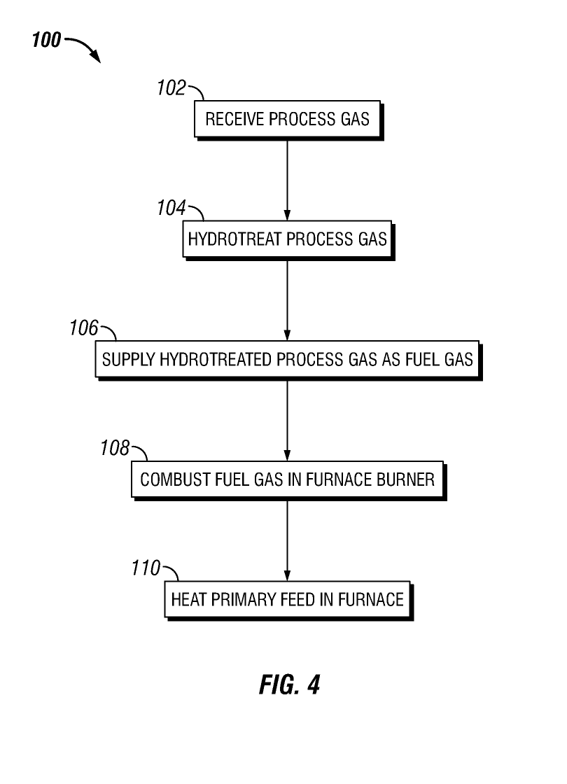

FIG. 4 is a method of operating a furnace system 100, such as a pyrolysis furnace used to produce olefins, a furnace in refinery applications, or a steam boiler, and so on. Process gas is received (block 102) into a fuel gas system that supplies fuel gas to burners of the furnace. The process gas is hydrotreated (block 104) to hydrogenate (saturate) unsaturated hydrocarbons (e.g., olefins, diolefins, and acetylenes) in the process gas. Examples of unsaturated hydrocarbons in the process gas to be hydrogenated include ethylene, acetylene, propylene, methyl-acetylenes, butenes, vinyl-actylenes, butadiene, and so forth. The hydrotreating, as indicated by reference numeral 104, thus may convert ethylene, propylene, and butenes in the process gas to ethane, propane, and butanes, respectively, for example. Likewise, the hydrogenation may convert acetylene and methyl-acetylene into ethane and propane, respectively.

The hydrotreated process gas having reduced or no unsaturated hydrocarbons may be supplied (block 106) as fuel gas to burners of the furnace. The fuel gas is combusted (block 108) at the burners in the furnace. The combustion may be facilitated with the addition and mixing of air or oxygen with the fuel gas. The burner flame of the combustion may heat (block 110) the primary feed in the furnace.

In sum, the embodiments of the present techniques may provide an olefin production system including a pyrolysis furnace to thermally crack hydrocarbon feedstock into olefins, and a fuel gas system having a hydrotreater to hydrogenate process gas (e.g., vent gas or process waste gas having unsaturated hydrocarbons, hydrogen, olefins or diolefins, or a combination thereof, etc.) for fuel gas supply to burners of the pyrolysis furnace. The fuel gas system receives the process gas from a refinery, a chemical plant, a storage system, process equipment downstream of the pyrolysis furnace, or any combination thereof. The fuel gas system receives the process gas, hydrogenates unsaturated compounds in the process gas via the hydrotreater, and supplies the hydrogenated process gas as fuel gas to the burners. The fuel gas system may further supply natural gas as fuel gas to the burners.

Similarly, the techniques disclosed herein may also provide a method of producing olefins, the method including receiving feedstock (e.g., from a refinery) and thermally cracking the feedstock in a pyrolysis furnace to give olefins, and receiving process gas (e.g., from a refinery) and hydrotreating the process gas for supply as fuel gas to a burner of the pyrolysis furnace. The process gas may be vent gas and/or waste gas from a refinery or chemical plant. Hydrotreating the process gas may include routing the process gas through hydrogenation catalyst. Further, the method may include supplying both natural gas and the hydrotreated process gas as fuel gas to the burner of the pyrolysis furnace.

Furthermore, other embodiments of the present techniques may provide for a furnace system including a furnace, and a hydrotreater to hydrogenate unsaturated compounds in fuel gas supplied to a burner of the furnace. Examples of furnace applications include steam boiler, electricity generator, olefin production, refinery treatment, and so on. The hydrotreater may hydrogenate at least olefins and diolefins in the fuel gas, and convert the olefins and diolefins in the fuel gas into alkanes. The furnace system includes a process gas collection system disposed upstream of the hydrotreater. The furnace system may include a process gas header disposed upstream of the hydrotreater and configured to receive process gas to be sent through the hydrotreater as fuel gas to the burner.

Lastly, embodiments of the present techniques may provide a fuel system for one or more furnaces, including a process fluid header configured to receive process fluid, and a hydrotreater reactor configured to hydrogenate unsaturated hydrocarbons in the process fluid to give a treated process fluid. The fuel system supplies the treated process fluid as fuel to a burner of a furnace. The hydrotreater reactor contains hydrogenation catalyst. The fuel system may additionally provide natural gas as fuel to the burner.

This concludes the detailed description. The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. An olefin production system comprising:
a pyrolysis furnace configured to thermally crack hydrocarbon feedstock into olefins, the pyrolysis furnace having a burner; and
a fuel gas system comprising a hydrotreater system configured to hydrogenate process gas, the fuel gas system configured to supply the hydrogenated process gas as fuel gas to the burner.

2. The olefin production system of claim 1, wherein the process gas comprises unsaturated hydrocarbons.

3. The olefin production system of claim 2, wherein the process gas further comprises hydrogen.

4. The olefin production system of claim 1, wherein the process gas is vent gas or waste gas, or a combination thereof.

5. The olefin production system of claim 1, wherein the fuel gas system is configured to receive the process gas from a refinery, a chemical plant, a storage system, process equipment downstream of the pyrolysis furnace, or any combination thereof.

6. The olefin production system of claim 1, wherein the process gas comprises olefins or diolefins, or a combination thereof.

7. The olefin production system of claim 1, wherein the fuel gas system is configured to:
receive the process gas;
hydrogenate unsaturated compounds in the process gas via the hydrotreater system; and
supply the hydrogenated process gas as fuel gas to the burner.

8. The olefin production system of claim 1, wherein the fuel gas system is further configured to supply natural gas as fuel gas to the burner.

9. A furnace system comprising:
a furnace including a burner; and
a hydrotreater system to hydrogenate unsaturated compounds in fuel gas supplied to the burner of the furnace.

10. The furnace system of claim 9, wherein the hydrotreater system is configured to:
hydrogenate olefins and diolefins in the fuel gas, and
convert the olefins and diolefins in the fuel gas into alkanes.

11. The furnace system of claim 9, comprising a process gas collection system disposed upstream of the hydrotreater system.

12. The furnace system of claim 9, comprising a process gas header disposed upstream of the hydrotreater system and configured to receive process gas to be sent through the hydrotreater system as fuel gas to the burner.

13. A method of producing olefins, the method comprising:
receiving feedstock;
thermally cracking the feedstock in a pyrolysis furnace to give olefins;
receiving process gas; and
hydrotreating the process gas for supply as fuel gas to a burner of the pyrolysis furnace.

14. The method of claim 13, wherein the process gas comprises at least one of vent gas or waste gas from a refinery or a chemical plant.

15. The method of claim 13, wherein:
receiving feedstock comprises receiving the feedstock from a refinery; and
receiving process gas comprises receiving process gas from the refinery.

16. The method of claim 13, wherein hydrotreating the process gas comprises routing the process gas through a bed of hydrogenation catalyst.

17. The method of claim 13, comprising supplying both natural gas and the hydrotreated process gas as fuel gas to the burner of the pyrolysis furnace.

18. A fuel system comprising:
a process fluid header configured to receive process fluid; and
a hydrotreater reactor configured to hydrogenate unsaturated hydrocarbons in the process fluid to give a treated process fluid, wherein the fuel system is configured to supply the treated process fluid as fuel to a burner of a furnace.

19. The fuel system of claim 18, wherein the hydrotreater reactor comprises hydrogenation catalyst.

20. The fuel system of claim 18, wherein the fuel system is further configured to additionally provide natural gas as fuel to the burner.

21. The fuel system of claim 18, wherein the fuel system is configured to supply the treated process fluid as fuel to a plurality of furnaces.
